(19) United States
(12) Patent Application Publication
(10) Pub. No.: US 2009/0202626 A1
(43) Pub. Date: Aug. 13, 2009

(54) TREATMENT OF BLADDER DISEASES WITH A TLR7 ACTIVATOR

(76) Inventors: Dennis A. Carson, La Jolla, CA (US); Lorenzo M. Leoni, Lodrino (CH)

Correspondence Address: SCHWEGMAN, LUNDBERG & WOESSNER, P.A., P.O. BOX 2938, MINNEAPOLIS, MN, 55402, US

(21) Appl. No.: 12/367,172

(22) Filed: Feb. 6, 2009

Related U.S. Application Data
(60) Provisional application No. 61/026,999, filed on Feb. 7, 2008.

(51) Int. Cl. A61K 9/127; A61K 38/00; A61K 31/4745

(52) U.S. Cl. 424/450; 514/12; 514/293

(57) ABSTRACT

The invention provides a method for the treatment of superficial bladder cancer and inflammatory diseases of the bladder which employs certain Toll-like Receptor (TLR)-agonists.

### CROSS-REFERENCE TO RELATED APPLICATIONS

This application claims the benefit of the filing date of U.S. Provisional Application Ser. No. 61/026,999, filed on Feb. 7, 2008, the disclosure of which is incorporated by reference herein.

### STATEMENT OF GOVERNMENT RIGHTS

The invention was made, at least in part, with a grant, from the Government of the United States of America (grant AI050564 from the National Institute of Allergy and Infectious Diseases). The Government has certain rights in the invention.

### BACKGROUND

A great deal has been learned about the molecular basis of innate recognition of microbial pathogens in the last decade. It is generally accepted that many somatic cells express a range of pattern recognition receptors that detect potential pathogens independently of the adaptive immune system (Janeway et al., 2002). These receptors are believed to interact with microbial components termed pathogen associated molecular patterns (PAMPs). Examples of PAMPs include peptidoglycans, lipotechoic acids from gram-positive cell walls, the sugar mannose (which is common in microbial carbohydrates but rare in humans), bacterial DNA, double-stranded RNA from viruses, and glucans from fungal cell walls. PAMPs generally meet certain criteria that include (a) their expression by microbes but not their mammalian hosts, (b) conservation of structure across the wide range of pathogens, and (c) the capacity to stimulate innate immunity.

Toll-like Receptors (TLRs) have been found to play a central role in the detection of PAMPs and in the early response to microbial infections (Underhill et al., 2002). Ten mammalian TLRs and a number of their agonists have been identified. For example, TLR7 and TLR9 recognize and respond to imiquimod and immunostimulatory CpG oligonucleotides (ISS-ODN), respectively. The synthetic immunomodulator R-848 (resiquimod) activates both TLR7 and TLR8.

The discovery that endogenous ligands as well as synthetic small molecules can activate certain TLR pathways has generated interest in the development of new therapeutics for diseases related to the immune response. TLR ligands control the activation of antigen-presenting cells, in particular dendritic cells, by triggering their maturation program, including up-regulation of the expression of HLA and costimulatory molecules and secretion of proinflammatory cytokines, such as TNF-α, IL-6, IL-12, and IFN-α (Stanley, 2002).

While TLR stimulation initiates a common signaling cascade (involving the adaptor protein MyD88, the transcription factor NF-kB, and pro-inflammatory and effector cytokines), certain cell types tend to produce certain TLRs. For example, TLR7 and TLR9 are found predominantly on the internal faces of endosomes in dendritic cells (DCs) and B lymphocytes (in humans; mouse macrophages express TLR7 and TLR9). TLR8, on the other hand, is found in human blood monocytes (Hornung et al., 2002).

While agonists of TLRs have great therapeutic potential, their utility has been limited by side effects related to the release and systemic dispersion of proinflammatory cytokines. Therefore, the major in vivo applications of TLR7 ligands have been as topically applied antiviral or antitumor agents or as immune adjuvants injected intramuscularly in small quantities (Ambach et al., 2004; Hemmi et al., 2002).

### SUMMARY OF THE INVENTION

The invention provides a method for the treatment of superficial bladder cancer and inflammatory diseases of the bladder, e.g., interstitial cystitis or overactive bladder. The method includes the administration of a synthetic TLR7 activator (agonist) formulated to optimize concentration of the synthetic TLR7 agonist in the bladder mucosa versus the blood, modified to optimize concentration of the synthetic TLR7 agonist in the bladder mucosa versus the blood, or co-administered with another treatment to optimize concentration of the synthetic TLR7 agonist in the bladder mucosa versus the blood. For example, the synthetic TLR7 agonist is formulated, modified or administered in conjunction with another treatment, so as to achieve a bladder mucosal concentration at least 2, 5, or more, e.g., at least 10, times higher than in the blood For example, if concentrations of the TLR7 agonist in the blood are generally in the range of about 10 nM to about 1000 nM, concentrations in the bladder are about 100 nM to about 10,000 nM. In one embodiment, the TLR7 agonist is administered in conjunction with locally applied ultrasound, electromagnetic radiation or electroporation or other electrically based drug delivery techniques, local chemical abrasion, or local physical abrasion, to disrupt the bladder permeability barrier. In one embodiment, the TLR7 agonist is administered with a locally applied surfactant to enhance permeability of the TLR7 agonist across the bladder mucosa. In one embodiment, the TLR agonist, a formulation thereof, or a conjugate thereof has enhanced endosomal uptake, for instance, as a result of particle size, induces receptor multimerization, and/or provides for sustained release. In particular, local activation of TLR7 may disrupt the cancer cell-matrix interactions that are required for growth and survival of malignant cells and may induce apoptosis.

In one embodiment, the formulation or conjugate has enhanced potency versus a corresponding TLR7 agonist (not formulated or conjugated), e.g., as determined in vitro or in vivo by cytokine induction assays, low systemic distribution, e.g., as determined using in vivo animal models and intravesical or other local delivery, and/or an improved activity/safety ratio, determined using in vivo animal models and intravesical or other local delivery.

In one embodiment, the TLR7 agonist may be formulated or chemically modified so as to minimize systemic absorption, e.g., by dispersion in emulsions, encapsulation in nanoparticles or lipsomes, aggregation in nanoparticles or nanocrystals, or chemical tethering to a protein or lipid (see, e.g., U.S. application Ser. Nos. 60/710,337; 60/809,870; 60/809,879; and 10/824,833, which are incorporated by reference herein).

In one embodiment, a TLR7 agonist for use in the invention has formula I:

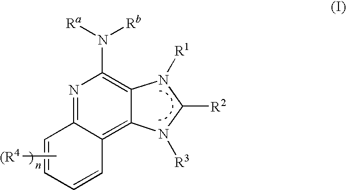

wherein

R¹, R², and R³ are each independently hydrogen; cyclic alkyl of three, four, or five carbon atoms; straight chain or branched chain alkyl containing one to about ten carbon atoms and substituted straight chain or branched chain alkyl containing one to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; fluoro- or chloroalkyl containing from one to about ten carbon atoms and one or more fluorine or chlorine atoms; straight chain or branched chain alkenyl containing two to about ten carbon atoms and substituted straight chain or branched chain alkenyl containing two to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; hydroxyalkyl of one to about six carbon atoms; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms, with the proviso that any such alkyl, substituted alkyl, alkenyl, substituted alkenyl, hydroxyalkyl, alkoxyalkyl, or acyloxyalkyl group does not have a fully carbon substituted carbon atom bonded directly to the nitrogen atom; benzyl; (phenyl)ethyl; and phenyl; said benzyl, (phenyl)ethyl or phenyl substituent being optionally substituted on the benzene ring by one or two moieties independently selected from the group consisting of alkyl of one to about four carbon atoms, alkoxy of one to about four carbon atoms, and halogen, with the proviso that when said benzene ring is substituted by two of said moieties, then the moieties together contain no more than six carbon atoms; —CHRₓR_{y} wherein R_{y} is hydrogen or a carbon-carbon bond, with the proviso that when R_{y} is hydrogen Rₓ is alkoxy of one to about four carbon atoms, hydroxyalkoxy of one to about four carbon atoms, 1-alkynyl of two to about ten carbon atoms, tetrahydropyranyl, alkoxyalkyl wherein the alkoxy moiety contains one to about four carbon atoms and the alkyl moiety contains one to about four carbon atoms, 2-, 3-, or 4-pyridyl, and with the further proviso that when R_{y} is a carbon-carbon bond R_{y} and Rₓ together form a tetrahydrofuranyl group optionally substituted with one or more substituents independently selected from the group consisting of hydroxy or hydroxyalkyl of one to about four carbon atoms;

straight chain or branched chain alkyl containing one to about eight carbon atoms, straight chain or branched chain hydroxyalkyl containing one to about six carbon atoms, morpholinomethyl, benzyl, (phenyl)ethyl and phenyl, the benzyl, (phenyl)ethyl or phenyl substituent being optionally substituted on the benzene ring by a moiety selected from the group consisting of methyl, methoxy, or halogen; or

—C(R_{S})(R_{T})(X) wherein R_{S} and R_{T} are independently selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen; and

X is alkoxy containing one to about four carbon atoms, alkoxyalkyl wherein the alkoxy moiety contains one to about four carbon atoms and the alkyl moiety contains one to about four carbon atoms, haloalkyl of one to about four carbon atoms, alkylamido wherein the alkyl group contains one to about four carbon atoms, amino, substituted amino wherein the substituent is alkyl or hydroxyalkyl of one to about four carbon atoms, azido, alkylthio of one to about four carbon atoms, or morpholinoalkyl wherein the alkyl moiety contains one to about four carbon atoms;

R⁴ is hydrogen, C₁₋₈ alkyl, C₁₋₈ alkoxy, or halo;

n is 1, 2, 3, or 4;

R^{a} and R^{b} are each independently hydrogen, (C₁-C₆)alkyl, hydroxy(C₁-C₆)alkyl, adamantyl, adamantyl(C₁-C₆)alkyl, amino(C₁-C₆)alkyl, aminosulfonyl, (C₁-C₆)alkanoyl, aryl, or benzyl; or R^{a} and R^{b} together with the nitrogen to which they are attached form a pyrrolidino, piperidino, or morpholino group; and

the dashed lines in the five membered ring of formula I denote an optional bond that connects a nitrogen of the five membered ring to the carbon that is between the two nitrogens of the five membered ring, and when the bond is present, either R¹ or R³ is absent;

or a pharmaceutically acceptable salt thereof.

In one embodiment, the synthetic TLR agonist conjugates for use in the methods of the invention are those disclosed in PCT/US06/032371, the disclosure of which is incorporated by reference herein. In one embodiment, a TLR agonist conjugates for use in the methods of the invention is a compound of formula (IC):

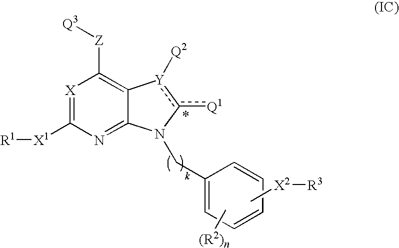

wherein

X is N or CR^{x} wherein R^{x} is hydrogen, halogen, substituted alkyl, unsubstituted alkyl, substituted heteroalkyl, or unsubstituted heteroalkyl;

Y is S or N;

the dashes (----) indicate optional bonds; wherein:

when the bond between Y and the carbon marked by an asterisk is a double bond, Q² is not present;

when the bond between Q¹ and the carbon marked by an asterisk is a double bond, Q¹ is O, S, NY¹, or NNY²Y³; and

when the bond between Q¹ and the carbon marked by an asterisk is a single bond, Q¹ is hydrogen, cyano, nitro, O—Y², S—Y², NY¹Y², or NY² NY³Y⁴; wherein

Y¹ is hydrogen, substituted alkyl, unsubstituted alkyl, substituted cycloalkyl, unsubstituted cycloalkyl, substituted heteroalkyl, unsubstituted heteroalkyl, substituted aryl, unsubstituted aryl, substituted heteroaryl, unsubstituted heteroaryl, —C(═O)— substituted alkyl, —C(═O)— unsubstituted alkyl, —C(═O)O— substituted alkyl, —C(═O)O— unsubstituted alkyl, cyano, nitro, hydroxyl, or O—Y²;

Y², Y³, and Y⁴, are each independently hydrogen, substituted alkyl, unsubstituted alkyl, substituted heteroalkyl, unsubstituted heteroalkyl, substituted aryl, unsubstituted aryl, substituted heteroaryl, unsubstituted heteroaryl;

Z is O, S, or NY⁵ wherein Y⁵ is hydrogen, substituted alkyl, unsubstituted alkyl, substituted heteroalkyl, unsubstituted heteroalkyl, substituted aryl, unsubstituted aryl, substituted heteroaryl, unsubstituted heteroaryl;

Q² and Q³ are each independently hydrogen, substituted alkyl, unsubstituted alkyl, substituted heteroalkyl, unsubstituted heteroalkyl, substituted aryl, unsubstituted aryl, substituted heteroaryl, unsubstituted heteroaryl;

X¹ is —O—, —S—, or —NR^{c}—;

R^{c} is hydrogen, C₁₋₁₀alkyl, or substituted C₁₋₁₀alkyl, or R^{c} and R¹ taken together with the nitrogen atom can form a heterocyclic ring or a substituted heterocyclic ring;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, or substituted C₅₋₉heterocyclic ring;

each R² is independently hydrogen, —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), —O—C(O)NR^{a}R^{b}, —(C₁-C₆)alkylene-NR^{a}R^{b}, —(C₁-C₆)alkylene-C(O)NR^{a}R^{b}, halo, nitro, or cyano;

each R^{a} and R^{b} is independently hydrogen, (C₁-C₆)alkyl, (C₃-C₈)cycloalkyl, (C₁-C₆)heteroalkyl, (C₁-C₆)alkoxy, halo(C₁-C₆)alkyl, (C₃-C₈)cycloalkyl(C₁-C₆)alkyl, (C₁-C₆)alkanoyl, hydroxy(C₁-C₆)alkyl, aryl, aryl(C₁-C₆)alkyl, Het, Het (C₁-C₆)alkyl, or (C₁-C₆)alkoxycarbonyl;

wherein the substituents on any alkyl, cycloalkyl, heteroalkyl, amino, alkoxy, alkanoyl, aryl, heteroaryl, or heterocyclic groups are one or more (e.g., 1, 2, 3, 4, 5, or 6) hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halogen, heterocycle (such as piperidinyl or morpholinyl), or aryl;

X² is a bond or a linking group;

k is 0, 1, 2, 3, or 4;

n is 0, 1, 2, 3, or 4; and

R³ is a macromolecule comprising a cell, virus, vitamin, cofactor, peptide, protein, nucleic acid molecule, lipid, bead or particle, such as a polystyrene bead or nanoparticles, or a dendrimer;

or a pharmaceutically acceptable salt thereof, including hydrates thereof.

In one embodiment, the synthetic TLR7 agonist for use in the methods of the invention include formulations or modifications of imiquimod, e.g., TMX 101, resiquimod, bropirimine, propirimine, or other TLR7 agonists, such as those described in U.S. Pat. No. 6,329,381 and Lee et al., Proc. Natl. Acad. Sci. USA, 103:1828 (2006), e.g., (9-benzyl-8-hydroxy-2-(2-methoxyethoxy)adenine), the disclosures of which are incorporated by reference herein, or co-treatments that include imiquimod or resiquimod administration.

In addition, the invention also provides a pharmaceutical composition comprising at least one compound of the invention, or a pharmaceutically acceptable salt thereof, in combination with a pharmaceutically acceptable diluent or carrier. Further, the invention provides a pharmaceutical composition comprising the compounds disclosed herein in combination with other known anticancer compounds.

In one embodiment, the invention provides a method to inhibit or treat a bladder, cervical, lung or anal disorder in a mammal, e.g., a human patient, by administering an effective amount of a TLR7 agonist that is modified or formulated, or administered in conjunction with another treatment. Patients to be treated include but are not limited to those with non-invasive bladder cancer, interstitial cystitis, cervical dysplasia, metastatic lung cancer, relapsed/refractory superfacial bladder cancer, and anal intra-epithelial neoplasia, or any preneoplastic or neoplastic condition that is accessible to local administration of a therapeutic agent, such as by direct application or use of a catheter or other drug delivery device. For instance, interstitial cystitis is common clinical syndrome in females characterized by frequency and dysuria. In some patients, the bladder is infiltrated with mast cells, and the urine has increased substance P, suggesting an allergic component. Stratification of patients may allow for a targeted treatment of a specific TLR7 agonist for interstitial cystitis.

The invention also provides a method to enhance killing of tumor cells in a mammal in need of such therapy. The method includes locally administering an effective amount of a compound of the invention to the mammal.

The present invention also provides a method for treating bladder, cervical, lung or anal cancer in a mammal, e.g., a human patient. The method includes locally contacting the cancer cells with a compound of the invention, or mixtures thereof, in an effective amount.

In addition, the present invention provides a method for inducing apoptosis or inducing cell death in cells in a mammal, e.g., a human patient. The method includes contacting target cells locally in vivo with a compound of the invention, or mixtures thereof, in an amount effective to enhance apoptosis or cell death in the target cells.

Thus, the invention provides compounds for use in medical therapy, such as agents that induce apoptosis or agents that inhibit or treat certain types of cancer, optionally in conjunction with other compounds. Accordingly, the compounds of the invention are useful to inhibit or treat cancer. Also provided is the use of the compounds for the manufacture of a medicament to enhance apoptosis or to inhibit or treat certain types of cancer.

### BRIEF DESCRIPTION OF THE FIGURES

FIG. 1. Exemplary TLR7 agonists.

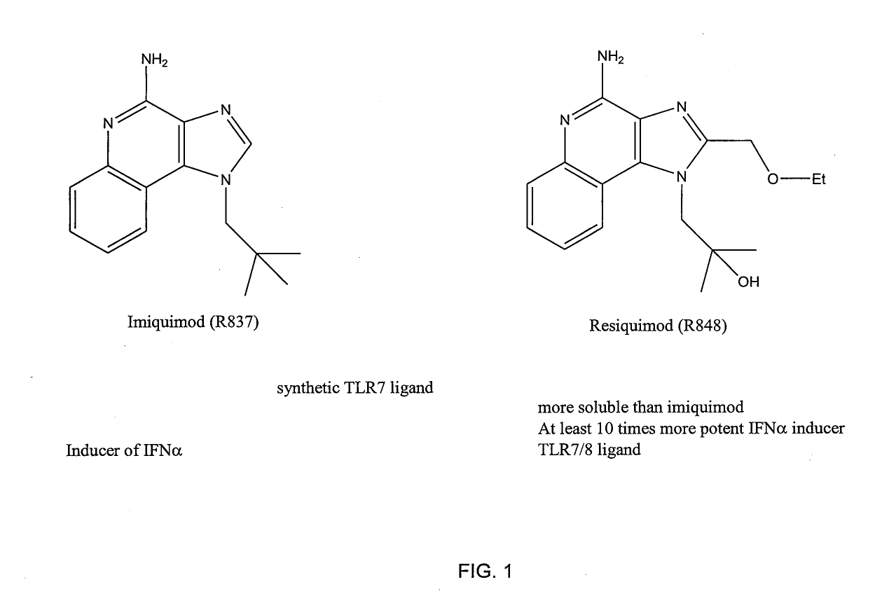

### DETAILED DESCRIPTION OF THE INVENTION

### Definitions

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

The term “amino acid” as used herein, comprises the residues of the natural amino acids (e.g. Ala, Arg, Asn, Asp, Cys, Glu, Gln, Gly, H is, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, -methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an -methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a compound of formula I through the carboxy terminus, the amino terminus, or through any other convenient point of attachment, such as, for example, through the sulfur of cysteine.

The term “toll-like receptor” (TLR) refers to a member of a family of receptors that bind to pathogen associated molecular patterns (PAMPs) and facilitate an immune response in a mammal. Ten mammalian TLRs are known, e.g., TLR1-10.

The term “toll-like receptor agonist” (TLR agonist) refers to a molecule that binds to a TLR and antagonizes the receptor. Synthetic TLR agonists are chemical compounds that are designed to bind to a TLR and activate the receptor. Exemplary novel TLR agonists provided herein include “TLR-7 agonist” “TLR-3 agonist” and “TLR-9 agonist.”

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

“Therapeutically effective amount” is intended to include an amount of a compound useful in the present invention or an amount of the combination of compounds claimed, e.g., to treat or prevent the disease or disorder, or to treat the symptoms of the disease or disorder, in a host. As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the methods of the present invention. Such organisms include, but are not limited to, mammals such as humans. In the context of the invention, the term “subject” generally refers to an individual who will receive or who has received treatment (e.g., administration of a compound of the invention, and optionally one or more anticancer agents) for cancer.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

### Methods and Compounds for Use in the Methods of the Invention

Bladder cancer has the 4th highest prevalence and the 5th highest incidence of all cancers in the U.S. and Europe. Every year in the United States more than 60,000 people are newly diagnosed with bladder cancer. The number of diagnosed bladder cancer patients has risen by more than 20% in the past decade, helped by effective diagnostic methods and the increase in the elderly population. 70% of bladder tumors are non-muscle invasive (superficial) at time of diagnosis, and 70% recur after initial transurethral resection.

The current standard-of-care for non-invasive bladder cancer is Bacille-Calmette-Guerin (BCG), a live attenuated mycobacteria, which is administered locally (intravesical) (80% of cases). BCG is an uncharacterized product, composed of an attenuated form of the bacterium Mycobacterium tuberculosis, used to prevent tuberculosis. BCG establishes a localized infection by attachment to and internalization in urothelium, which in turn releases IL-1, IL-6, and IL-8 (Hedges et al., 1994). Instillation of BCG results in an influx of neutrophils, followed by an influx of mononuclear cells consisting primarily of CD4⁺ cells. The net effect of chemokine signals is escalating recruitment of neutrophils and monocytic leukocytes into the bladder with each successive BCG instillation (Shapiro et al., 1988).

While there is a high incidence of complete local responses (70-75%) compared to intravesical chemotherapy, many patients ultimately need cystectomy due to recurrence and/or side effects and there are increased toxic side effects (local and systemic). For example, at least 30% of patients need to delay or stop BCG therapy due to local or systemic toxicity. Many clinicians are reluctant to use BCG because of the risks of life-threatening systemic infection/sepsis.

And although BCG has also been used for the treatment of interstitial cystitis, yielding a p value of 0=0.06 in a controlled trial, the infectious complications and systemic side effects of BCG administration may outweigh its value for noncancer related disorders such as interstitial cystitis.

The present invention provides for a locally administered TLR7 agonist, formulated in such a way that tissue penetration is promoted and systemic absorption is inhibited or prevented. Such a treatment is likely equally or more effective than BCG and without the systemic side effects of the live bacteria. For example, an in vivo mouse orthotopic bladder cancer transplantation model demonstrated that local TLR7 (intravesicular) activation with a conjugate of a TLR7 agonist did not result in systemic side effects and likely showed anti-tumor effects. In addition, in vivo efficacy of TLR7 agonist was demonstrated in bladder cancer cell lines by decreasing cell viability, inducing apoptosis and increasing cytokine production, which indicate that TLR7 agonists have anti-tumor effects. Activation of TLR7 may disrupt the interaction of the bladder cancer cells with growth factors bound to the extracellular matrix, which in turn may lead to apoptosis.

In one embodiment, the invention provides for treatment of established, superficial bladder cancer by intravesicular (in the bladder) administration of a synthetic TLR7 agonist, formulated or modified chemically so that it will achieve a maximal (local) concentration in the bladder mucosa, e.g., a concentration at least 10× higher than in the blood. To promote penetration, the TLR7 agonist may be combined with a physical or chemical treatment to disrupt the bladder permeability barrier, including locally applied ultrasound, all types of electromagnetic radiation, chemical and physical abrasion, and the use of surfactant. Inflammatory diseases of the bladder, including interstitial cystitis and overactive bladder, may be treated similarly.

The present TLR7 agonists are likely more potent and less toxic than BCG, and so achieve a more significant therapeutic effect. In one embodiment, the TLR7 agonist is administered to patients with a mast cell component to their disease, as indicated by biopsy of the bladder with histologic examination, and/or by measurement of elevated neurokinin levels (substance P) in the urine, in an amount effective to decrease mast cell function.

In one embodiment, the TLR7 agonist has formula I:

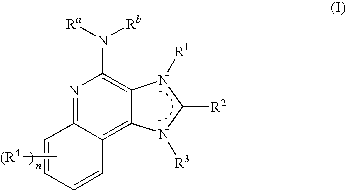

wherein

R¹, R², and R³ are each independently hydrogen; cyclic alkyl of three, four, or five carbon atoms; straight chain or branched chain alkyl containing one to about ten carbon atoms and substituted straight chain or branched chain alkyl containing one to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; fluoro- or chloroalkyl containing from one to about ten carbon atoms and one or more fluorine or chlorine atoms; straight chain or branched chain alkenyl containing two to about ten carbon atoms and substituted straight chain or branched chain alkenyl containing two to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; hydroxyalkyl of one to about six carbon atoms; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms, with the proviso that any such alkyl, substituted alkyl, alkenyl, substituted alkenyl, hydroxyalkyl, alkoxyalkyl, or acyloxyalkyl group does not have a fully carbon substituted carbon atom bonded directly to the nitrogen atom; benzyl; (phenyl)ethyl; and phenyl; said benzyl, (phenyl)ethyl or phenyl substituent being optionally substituted on the benzene ring by one or two moieties independently selected from the group consisting of alkyl of one to about four carbon atoms, alkoxy of one to about four carbon atoms, and halogen, with the proviso that when said benzene ring is substituted by two of said moieties, then the moieties together contain no more than six carbon atoms; —CHRₓR_{y} wherein R_{y} is hydrogen or a carbon-carbon bond, with the proviso that when R_{y} is hydrogen Rₓ is alkoxy of one to about four carbon atoms, hydroxyalkoxy of one to about four carbon atoms, 1-alkynyl of two to about ten carbon atoms, tetrahydropyranyl, alkoxyalkyl wherein the alkoxy moiety contains one to about four carbon atoms and the alkyl moiety contains one to about four carbon atoms, 2-, 3-, or 4-pyridyl, and with the further proviso that when R_{y} is a carbon-carbon bond R_{y} and R^{x} together form a tetrahydrofuranyl group optionally substituted with one or more substituents independently selected from the group consisting of hydroxy or hydroxyalkyl of one to about four carbon atoms;

straight chain or branched chain alkyl containing one to about eight carbon atoms, straight chain or branched chain hydroxyalkyl containing one to about six carbon atoms, morpholinomethyl, benzyl, (phenyl)ethyl and phenyl, the benzyl, (phenyl)ethyl or phenyl substituent being optionally substituted on the benzene ring by a moiety selected from the group consisting of methyl, methoxy, or halogen; or

—C(R_{S})(R_{T})(X) wherein R_{S} and R_{T} are independently selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen; and

X is alkoxy containing one to about four carbon atoms, alkoxyalkyl wherein the alkoxy moiety contains one to about four carbon atoms and the alkyl moiety contains one to about four carbon atoms, haloalkyl of one to about four carbon atoms, alkylamido wherein the alkyl group contains one to about four carbon atoms, amino, substituted amino wherein the substituent is alkyl or hydroxyalkyl of one to about four carbon atoms, azido, alkylthio of one to about four carbon atoms, or morpholinoalkyl wherein the alkyl moiety contains one to about four carbon atoms;

R⁴ is hydrogen, C₁₋₈ alkyl, C₁₋₈ alkoxy, or halo;

n is 1, 2, 3, or 4;

R^{a} and R^{b} are each independently hydrogen, (C₁-C₆)alkyl, hydroxy(C₁-C₆)alkyl, adamantyl, adamantyl(C₁-C₆)alkyl, amino(C₁-C₆)alkyl, aminosulfonyl, (C₁-C₆)alkanoyl, aryl, or benzyl; or R^{a} and R^{b} together with the nitrogen to which they are attached form a pyrrolidino, piperidino, or morpholino group; and

the dashed lines in the five membered ring of formula I denote an optional bond that connects a nitrogen of the five membered ring to the carbon that is between the two nitrogens of the five membered ring, and when the bond is present, either R₁ or R³ is absent;

or a pharmaceutically acceptable salt thereof.

In one embodiment, the TLR7 agonist includes imidazoquinoline amines such as 1H-imidazo[4,5-c]quinolin-4-amines as defined by one of Formulas II-VI below:

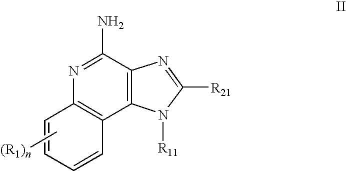

wherein

R₁₁, is selected from the group consisting of alkyl of one to about ten carbon atoms, hydroxyalkyl of one to about six carbon atoms, acyloxyalkyl wherein the acyloxy moiety is alkanoyloxy of two to about four carbon atoms or benzoyloxy, and the alkyl moiety contains one to about six carbon atoms, benzyl, (phenyl)ethyl and phenyl, said benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that if said benzene ring is substituted by two of said moieties, then said moieties together contain no more than six carbon atoms;

R₂₁ is selected from the group consisting of hydrogen, alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of alkyl of one to about four carbon atoms, alkoxy of one to about four carbon atoms and halogen, with the proviso that when the benzene ring is substituted by two of said moieties, then the moieties together contain no more than six carbon atoms; and

each R₁ is independently selected from the group consisting of alkoxy of one to about four carbon atoms, halogen, and alkyl of one to about four carbon atoms, and n is an integer from 0 to 2, with the proviso that if n is 2, then said R₁ groups together contain no more than six carbon atoms;

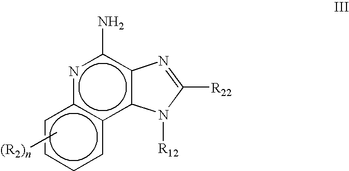

wherein

R₁₂ is selected from the group consisting of straight chain or branched chain alkenyl containing two to about ten carbon atoms and substituted straight chain or branched chain alkenyl containing two to about ten carbon atoms, wherein the substituent is selected from the group consisting of straight chain or branched chain alkyl containing one to about four carbon atoms and cycloalkyl containing three to about six carbon atoms; and cycloalkyl containing three to about six carbon atoms substituted by straight chain or branched chain alkyl containing one to about four carbon atoms; and

R₂₂ is selected from the group consisting of hydrogen, straight chain or branched chain alkyl containing one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl containing one to about four carbon atoms, straight chain or branched chain alkoxy containing one to about four carbon atoms, and halogen, with the proviso that when the benzene ring is substituted by two such moieties, then the moieties together contain no more than six carbon atoms; and

each R₂ is independently selected from the group consisting of straight chain or branched chain alkoxy containing one to about four carbon atoms, halogen, and straight chain or branched chain alkyl containing one to about four carbon atoms, and n is an integer from zero to 2, with the proviso that if n is 2, then said R₂ groups together contain no more than six carbon atoms;

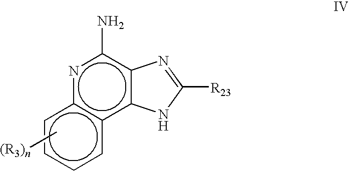

wherein

R₂₃ is selected from the group consisting of hydrogen, straight chain or branched chain alkyl of one to about eight carbon atoms, benzyl, (phenyl)ethyl and phenyl, the benzyl, (phenyl)ethyl or phenyl substituent being optionally substituted on the benzene ring by one or two moieties independently selected from the group consisting of straight chain or branched chain alkyl of one to about four carbon atoms, straight chain or branched chain alkoxy of one to about four carbon atoms, and halogen, with the proviso that when the benzene ring is substituted by two such moieties, then the moieties together contain no more than six carbon atoms; and

each R₃ is independently selected from the group consisting of straight chain or branched chain alkoxy of one to about four carbon atoms, halogen, and straight chain or branched chain alkyl of one to about four carbon atoms, and n is an integer from zero to 2, with the proviso that if n is 2, then said R₃ groups together contain no more than six carbon atoms;

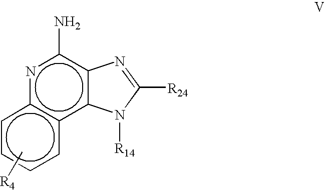

wherein

R₁₄ is —CHRₓR_{y} wherein R_{y} is hydrogen or a carbon-carbon bond, with the proviso that when R_{y} is hydrogen R^{x} is alkoxy of one to about four carbon atoms, hydroxyalkoxy of one to about four carbon atoms, 1-alkynyl of two to about ten carbon atoms, tetrahydropyranyl, alkoxyalkyl wherein the alkoxy moiety contains one to about four carbon atoms and the alkyl moiety contains one to about four carbon atoms, 2-, 3-, or 4-pyridyl, and with the further proviso that when R_{y} is a carbon-carbon bond R_{y} and R^{x} together form a tetrahydrofuranyl group optionally substituted with one or more substituents independently selected from the group consisting of hydroxy and hydroxyalkyl of one to about four carbon atoms;

R₂₄ is selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen; and

R₄ is selected from the group consisting of hydrogen, straight chain or branched chain alkoxy containing one to about four carbon atoms, halogen, and straight chain or branched chain alkyl containing one to about four carbon atoms;

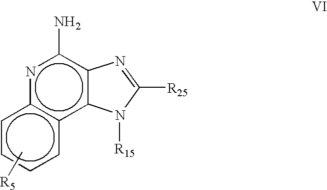

wherein

R₁₅ is selected from the group consisting of: hydrogen; straight chain or branched chain alkyl containing one to about ten carbon atoms and substituted straight chain or branched chain alkyl containing one to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; straight chain or branched chain alkenyl containing two to about ten carbon atoms and substituted straight chain or branched chain alkenyl containing two to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; hydroxyalkyl of one to about six carbon atoms; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms; benzyl; (phenyl)ethyl; and phenyl; said benzyl, (phenyl)ethyl or phenyl substituent being optionally substituted on the benzene ring by one or two moieties independently selected from the group consisting of alkyl of one to about four carbon atoms, alkoxy of one to about four carbon atoms, and halogen, with the proviso that when said benzene ring is substituted by two of said moieties, then the moieties together contain no more than six carbon atoms;

R₂₅ is

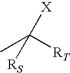

wherein

R_{S} and R_{T} are independently selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen;

X is selected from the group consisting of alkoxy containing one to about four carbon atoms, alkoxyalkyl wherein the alkoxy moiety contains one to about four carbon atoms and the alkyl moiety contains one to about four carbon atoms, hydroxyalkyl of one to about four carbon atoms, haloalkyl of one to about four carbon atoms, alkylamido wherein the alkyl group contains one to about four carbon atoms, amino, substituted amino wherein the substituent is alkyl or hydroxyalkyl of one to about four carbon atoms, azido, chloro, hydroxy, 1-morpholino, 1-pyrrolidino, alkylthio of one to about four carbon atoms; and

R₅ is selected from the group consisting of hydrogen, straight chain or branched chain alkoxy containing one to about four carbon atoms, halogen, and straight chain or branched chain alkyl containing one to about four carbon atoms;

or a pharmaceutically acceptable salt of any of the foregoing.

In one embodiment, the TLR7 agonist has formula VII below:

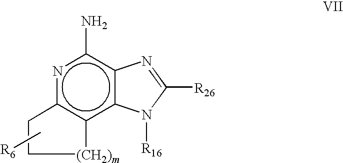

wherein m is 1, 2, or 3;

R₁₆ is selected from the group consisting of hydrogen; cyclic alkyl of three, four, or five carbon atoms; straight chain or branched chain alkyl containing one to about ten carbon atoms and substituted straight chain or branched chain alkyl containing one to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; fluoro- or chloroalkyl containing from one to about ten carbon atoms and one or more fluorine or chlorine atoms; straight chain or branched chain alkenyl containing two to about ten carbon atoms and substituted straight chain or branched chain alkenyl containing two to about ten carbon atoms, wherein the substituent is selected from the group consisting of cycloalkyl containing three to about six carbon atoms and cycloalkyl containing three to about six carbon atoms substituted by straight chain or branched chain alkyl containing one to about four carbon atoms; hydroxyalkyl of one to about six carbon atoms; alkoxyalkyl wherein the alkoxy moiety contains one to about four carbon atoms and the alkyl moiety contains one to about six carbon atoms; acyloxyalkyl wherein the acyloxy moiety is alkanoyloxy of two to about four carbon atoms or benzoyloxy, and the alkyl moiety contains one to about six carbon atoms, with the proviso that any such alkyl, substituted alkyl, alkenyl, substituted alkenyl, hydroxyalkyl, alkoxyalkyl, or acyloxyalkyl group does not have a fully carbon substituted carbon atom bonded directly to the nitrogen atom; benzyl; (phenyl)ethyl; and phenyl; said benzyl, (phenyl)ethyl or phenyl substituent being optionally substituted on the benzene ring by one or two moieties independently selected from the group consisting of alkyl of one to about four carbon atoms, alkoxy of one to about four carbon atoms, and halogen, with the proviso that when said benzene ring is substituted by two of said moieties, then the moieties together contain no more than six carbon atoms; and —CHRₓR_{y} wherein R_{y} is hydrogen or a carbon-carbon bond, with the proviso that when R_{y} is hydrogen R^{x} is alkoxy of one to about four carbon atoms, hydroxyalkoxy of one to about four carbon atoms, 1-alkynyl of two to about ten carbon atoms, tetrahydropyranyl, alkoxyalkyl wherein the alkoxy moiety contains one to about four carbon atoms and the alkyl moiety contains one to about four carbon atoms, 2-, 3-, or 4-pyridyl, and with the further proviso that when R_{y} is a carbon-carbon bond R_{y} and Rₓ together form a tetrahydrofuranyl group optionally substituted with one or more substituents independently selected from the group consisting of hydroxy and hydroxyalkyl of one to about four carbon atoms;

R₂₆ is selected from the group consisting of hydrogen, straight chain or branched chain alkyl containing one to about eight carbon atoms, straight chain or branched chain hydroxyalkyl containing one to about six carbon atoms, morpholinomethyl, benzyl, (phenyl)ethyl and phenyl, the benzyl, (phenyl)ethyl or phenyl substituent being optionally substituted on the benzene ring by a moiety selected from the group consisting of methyl, methoxy, and halogen; and

—C(R_{S})(R_{T})(X) wherein R_{S} and R_{T} are independently selected from the group consisting of hydrogen, alkyl of one to about four carbon atoms, phenyl, and substituted phenyl wherein the substituent is selected from the group consisting of alkyl of one to about four carbon atoms, alkoxy of one to about four carbon atoms, and halogen; and

X is selected from the group consisting of alkoxy containing one to about four carbon atoms, alkoxyalkyl wherein the alkoxy moiety contains one to about four carbon atoms and the alkyl moiety contains one to about four carbon atoms, haloalkyl of one to about four carbon atoms, alkylamido wherein the alkyl group contains one to about four carbon atoms, amino, substituted amino wherein the substituent is alkyl or hydroxyalkyl of one to about four carbon atoms, azido, alkylthio of one to about four carbon atoms, and morpholinoalkyl wherein the alkyl moiety contains one to about four carbon atoms, and

R₆ is selected from the group consisting of hydrogen, fluoro, chloro, straight chain or branched chain alkyl containing one to about four carbon atoms, and straight chain or branched chain fluoro- or chloroalkyl containing one to about four carbon atoms and at least one fluorine or chlorine atom;

or a pharmaceutically acceptable salt thereof.

In another embodiment, the TLR7 agonist has formula VIII below:

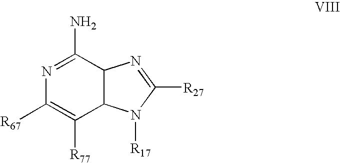

wherein

R₁₇ is selected from the group consisting of hydrogen; —CH₂R_{W} wherein R_{W} is selected from the group consisting of straight chain, branched chain, or cyclic alkyl containing one to about ten carbon atoms, straight chain or branched chain alkenyl containing two to about ten carbon atoms, straight chain or branched chain hydroxyalkyl containing one to about six carbon atoms, alkoxyalkyl wherein the alkoxy moiety contains one to about four carbon atoms and the alkyl moiety contains one to about six carbon atoms, and phenylethyl; and —CH==CR_{Z}R_{Z} wherein each R^{z} is independently straight chain, branched chain, or cyclic alkyl of one to about six carbon atoms;

R₂₇ is selected from the group consisting of hydrogen, straight chain or branched chain alkyl containing one to about eight carbon atoms, straight chain or branched chain hydroxyalkyl containing one to about six carbon atoms, alkoxyalkyl wherein the alkoxy moiety contains one to about four carbon atoms and the alkyl moiety contains one to about six carbon atoms, benzyl, (phenyl)ethyl and phenyl, the benzyl, (phenyl)ethyl or phenyl substituent being optionally substituted on the benzene ring by a moiety selected from the group consisting of methyl, methoxy, and halogen; and morpholinoalkyl wherein the alkyl moiety contains one to about four carbon atoms;

R₆₇ and R₇₇ are independently selected from the group consisting of hydrogen and alkyl of one to about five carbon atoms, with the proviso that R₆₇ and R₇₇ taken together contain no more than six carbon atoms, and with the further proviso that when R₇₇ is hydrogen then R₆₇ is other than hydrogen and R₂₇ is other than hydrogen or morpholinoalkyl, and with the further proviso that when R₆₇ is hydrogen then R₇₇ and R₂₇ are other than hydrogen;

and pharmaceutically acceptable salts thereof.

In another embodiment, the TLR7 agonist has formula IX below:

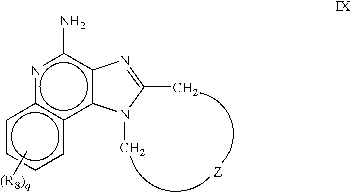

wherein

Z is selected from the group consisting of:

—(CH₂)ₚ— wherein p is 1 to 4;

—(CH₂)ₐ—C(R_{D}R_{E})(CH₂)_{b}—, wherein a and b are integers and a+b is 0 to 3, R_{D} is hydrogen or alkyl of one to four carbon atoms, and R_{E} is selected from the group consisting of alkyl of one to four carbon atoms, hydroxy, —OR_{F} wherein R_{F} is alkyl of one to four carbon atoms, and —NR_{G}R′_{G} wherein R_{G} and R′_{G} are independently hydrogen or alkyl of one to four carbon atoms; and

—(CH₂)ₐ—(Y)—(CH₂)_{b}— wherein a and b are integers and a+b is 0 to 3, and Y is O, S, or —NR_{J}— wherein R_{J} is hydrogen or alkyl of one to four carbon atoms;

and wherein q is 0 or 1 and R₈ is selected from the group consisting of alkyl of one to four carbon atoms, alkoxy of one to four carbon atoms, and halogen,

and pharmaceutically acceptable salts thereof.

The substituents R₁₁-R₁₇ above are generally designated “1-substituents” herein. In one embodiment, the 1-substituents are alkyl containing one to six carbon atoms and hydroxyalkyl containing one to six carbon atoms, e.g., the 1-substituent is 2-methylpropyl or 2-hydroxy-2-methylpropyl.

The substituents R₂₁-R₂₇ above are generally designated “2-substituents” herein. In one embodiment, the 2-substituents are hydrogen, alkyl of one to six carbon atoms, alkoxyalkyl wherein the alkoxy moiety contains one to four carbon atoms and the alkyl moiety contains one to four carbon atoms, and hydroxyalkyl of one to four carbon atoms, e.g., the 2-substituent is hydrogen, methyl, butyl, hydroxymethyl, ethoxymethyl or methoxyethyl.

In instances where n can be zero, one, or two, n is preferably zero or one.

The amounts of the compounds that will be therapeutically effective in a specific situation will of course depend on such things as the activity of the particular compound, the mode of administration, and the disease being treated. As such, it is not practical to identify specific administration amounts herein; however, those skilled in the art will be able to determine appropriate therapeutically effective amounts based on the guidance provided herein, information available in the art pertaining to these compounds, and routine testing.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine nicotine agonist activity using the standard tests described herein, or using other similar tests which are well known in the art.

In cases where compounds are sufficiently basic or acidic to form acid or base salts, use of the compounds as salts may be appropriate. Examples of acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Alkyl includes straight or branched C₁₋₁₀ alkyl groups, e.g., methyl, ethyl, propyl, butyl, pentyl, isopropyl, isobutyl, 1-methylpropyl, 3-methylbutyl, hexyl, and the like.

Lower alkyl includes straight or branched C₁₋₆ alkyl groups, e.g., methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, and the like.

The term “alkylene” refers to a divalent straight or branched hydrocarbon chain (e.g. ethylene —CH₂—CH₂—).

C₃₋₇ cycloalkyl includes groups such as, cyclopropyl, cyclopentyl, cyclohexyl, cycloheptyl, and the like, and alkyl-substituted C₃₋₇ cycloalkyl group, preferably straight or branched C₁₋₆ alkyl group such as methyl, ethyl, propyl, butyl or pentyl, and C₅₋₇ cycloalkyl group such as, cyclopentyl or cyclohexyl, and the like.

Lower alkoxy includes C₁₋₆ alkoxy groups, such as methoxy, ethoxy or propoxy, and the like.

Lower alkanoyl includes C₁₋₆ alkanoyl groups, such as formyl, acetyl, propanoyl, butanoyl, pentanoyl or hexanoyl, and the like.

C₇₋₁₁ aroyl, includes groups such as benzoyl or naphthoyl;

Lower alkoxycarbonyl includes C₂₋₇ alkoxycarbonyl groups, such as methoxycarbonyl, ethoxycarbonyl or propoxycarbonyl, and the like.

Lower alkylamino group means amino group substituted by C₁₋₆ alkyl group, such as, methylamino, ethylamino, propylamino, butylamino, and the like.

Di(lower alkyl)amino group means amino group substituted by the same or different and C₁₋₆ alkyl group (e.g. dimethylamino, diethylamino, ethylmethylamino).

Lower alkylcarbamoyl group means carbamoyl group substituted by C₁₋₆ alkyl group (e.g. methylcarbamoyl, ethylcarbamoyl, propylcarbamoyl, butylcarbamoyl).

Di(lower alkyl)carbamoyl group means carbamoyl group substituted by the same or different and C₁₋₆ alkyl group (e.g. dimethylcarbamoyl, diethylcarbamoyl, ethylmethylcarbamoyl).

Halogen atom means halogen atom such as fluorine atom, chlorine atom, bromine atom or iodine atom.

Aryl refers to a C₆₋₁₀ monocyclic or fused cyclic aryl group, such as phenyl, indenyl, or naphthyl, and the like.

Heterocyclic refers to monocyclic saturated heterocyclic groups, or unsaturated monocyclic or fused heterocyclic group containing at least one heteroatom, e.g., 0-3 nitrogen atoms, 0-1 oxygen atom (—O—), and 0-1 sulfur atom (—S—). Non-limiting examples of saturated monocyclic heterocyclic group includes 5 or 6 membered saturated heterocyclic group, such as tetrahydrofuranyl, pyrrolidinyl, morpholinyl, piperidyl, piperazinyl or pyrazolidinyl. Non-limiting examples of unsaturated monocyclic heterocyclic group includes 5 or 6 membered unsaturated heterocyclic group, such as furyl, pyrrolyl, pyrazolyl, imidazolyl, thiazolyl, thienyl, pyridyl or pyrimidinyl. Non-limiting examples of unsaturated fused heterocyclic groups includes unsaturated bicyclic heterocyclic group, such as indolyl, isoindolyl, quinolyl, benzothizolyl, chromanyl, benzofuranyl, and the like.

Alkyl, aryl, and heterocyclic groups can be optionally substituted with one or more substituents, wherein the substituents are the same or different, and include lower alkyl; C₁₋₆ alkoxy, such as methoxy, ethoxy or propoxy; carboxyl; C₂₋₇ alkoxycarbonyl, such as methoxycarbonyl, ethoxycarbonyl or propoxycarbonyl) and halogen; cycloalkyl and include C₃₋₆ cycloalkyl; hydroxyl; C₁₋₆ alkoxy; amino; cyano; aryl; substituted aryl, such as 4-hydroxyphenyl, 4-methoxyphenyl, 4-chlorophenyl or 3,4-dichlorophenyl; nitro and halogen, hydroxyl; hydroxy C₁₋₆ alkylene, such as hydroxymethyl, 2-hydroxyethyl or 3-hydroxypropyl; lower alkoxy; C₁₋₆ alkoxy C₁₋₆ alkyl, such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C₁₋₆ alkylthio, such as, methylthio, ethylthio, propylthio or butylthio; substituted C₁₋₆ alkylthio, such as methoxyethylthio, methylthioethylthio, hydroxyethylthio or chloroethylthio; aryl; substituted C₆₋₁₀ monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimino.

The heterocyclic ring can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano.

The compounds of the invention can be formulated as pharmaceutical compositions and administered to a mammalian host, such as a human patient in a variety of forms adapted to the chosen route of administration, e.g., orally or parenterally, by intravenous, intramuscular, topical or subcutaneous routes. In one embodiment, the composition is locally administered, e.g., intravesicularly.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may be administered by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms can include sterile aqueous solutions or dispersions or sterile powders comprising the active ingredient which are adapted for the extemporaneous preparation of sterile solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them as compositions or formulations, in combination with an acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949. The ability of a compound of the invention to act as a TLR agonist may be determined using pharmacological models which are well known to the art, including the procedures disclosed by Lee et al., PNAS, 100:6646 (2003).

Generally, the concentration of the compound(s) in a liquid composition will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.01 to about 100 μM, 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will be further described by the following non-limiting example.

### Example 1

The systemic delivery of TLR7 agonists is not ideal since it does not allow for the organization of the immune response in a particular part of the body. TLR7 agonists display the highest activity when delivered locally allowing the creation of a potent immune gradient. The localized delivery also reduces the risk of systemic exposure, thereby increasing the safety profile of the agonist. Bladder is an immunologically active organ, “skin turned inside out,” with TLR7 expressing dendritic and mast cells. To achieve good clinical activity for a bladder cancer patient, optimal passage of TLR7 agonists through the bladder permeability barrier is needed. Too great permeability leads to systemic side effects, while poor permeability leads to incomplete eradication. TLR7 agonist conjugates, e.g., conjugates of imiquimod, can improve the uptake of the agonist by enhancing adhesion, endosomal uptake, and/or receptor multimerization (reducing monomeric interactions), and may provide for sustained drug release to improve to duration of effect.

Bladder cancer patients amenable to treatment with a TLR7 agonist of the invention include, but are not limited to, those for whom most of the tumor has been removed by trans-urethral resection, but some residual cancer persists, and can be observed during cytoscopy, patients with high-risk and mid-risk non-muscle invasive bladder cancer and the patients with carcinoma in situ (cis) of the bladder. In one embodiment, the TLR7 agonist is formulated so as to minimize systemic absorption, e.g., via dispersion in emulsions, encapsulation in nanoparticles or lipsomes, aggregation in nanoparticles or nanocrystals, or chemical tethering to a protein or lipid. In one embodiment, the TLR7 formulations are administered via a catheter in the urethra, and the catheter is clamped to allow for drug contact with the cancer, e.g., for about 10 minutes to 2 hours after which the bladder is flushed to remove unreacted drug. The procedure may be repeated at approximately weekly intervals×6, and then monthly.

Exemplary conjugates are conjugates with propirimine or imiquimod. Bropirimine (a TLR agonist) has been shown to be effective in superficial bladder cancer (European Urology, Vol 34, 1998). Imiquimod has demonstrated efficacy in superficial skin cancer, inhibited chemically induced bladder cancer and cured mice of the FCB bladder tumor (Borden et al., 1990). Imiquimod also showed potent anti-tumor activity in an orthotopic bladder cancer mouse model (Smith et al., 2007). In placebo treated animals, 11 of 13 mice (85%) developed invasive, high-grade bladder tumors. In the imiquimod-treated animals (100 μg once weekly), only 3 of 14 mice developed tumors.

TMX-101 is a formulation of imiquimod designed to improve activity and retard systemic absorption. To determine the activity of TMX101 against superficial bladder cancer, TMX101 was delivered locally via intravesical instillation.

### SUMMARY

The main advantages of a better formulation, a better dosage or a better mode of delivery for a TLR7 agonist (such as imiquimod) in bladder diseases are:

1) reduced toxicity: by modifying the formulation or dosage of a TLR7 agonist, e.g., imiquimod, the local effect is maximized and the systemic exposure is reduced. This can be achieved using formulation techniques (such as the use of in situ forming gels or depots, in combination with excipients, use of lipids, and the like). The pharmacokinetic profile and the ratio between “bladder” versus “plasma” levels of “unformulated” TLR7 agonists versus formulations of TLR7 agonists is determined and formulations with improved profiles are selected for use in the methods of the invention;
2) improved efficacy: the efficacy of TLR7 molecules depends on the profile of cytokines/chemokines that can be triggered. The cytokine/chemokine profile can change based on how the TLR7 ligands enter the target cells, which endosomal compartment is activated, and other factors. The cytokine/chemokine profile of “unformulated” TLR7 agonists is different from that of the improved formulations or delivery systems. Formulations or delivery systems that provide the best efficacy in animal models of bladder cancer are selected for use in the methods of the invention;
3) better therapeutical window: the result of a better safety profile and increased efficacy provides a clear advantage over the “unformulated” TLR7 agonist.

### REFERENCES

Ambach et al., Mol. Immunol., 40:1307 (2004).
Borden et al., Cancer Res. 50:1071 (1990).
Hemmi et al., Nat. Immunol., 3:196 (2002).
Hornung et al., J. Immunol., 168:4531 (2002).
Janeway et al., Ann. Rev. Immunol. 20:197 (2002).
Shapiro et al., World. J. Urol., 6:61 (1988).
Smith et al., J. Urol., 177:2347 (2007).
Stanley, Clin. Exp. Dermatol., 27:571 (2002).
Underhill et al., Curr. Opin. Immunol., 14:103 (2002).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to inhibit or treat superficial bladder cancer in a mammal, comprising administering intravesicularly to a mammal having superficial bladder cancer an effective amount of a composition comprising a TLR7 agonist formulated or chemically modified to inhibit systemic adsorption or to enhance local concentrations of the agonist in the bladder mucosa.

2. The method of claim 1 wherein the composition comprises a chemically modified TLR7 agonist.

3. The method of claim 2 wherein the modification is the covalent linkage of the TLR7 agonist to a protein or a lipid.

4. The method of claim 1 wherein the composition comprises an emulsion.

5. The method of claim 1 wherein the composition comprises nanoparticles.

6. The method of claim 1 wherein the composition comprises liposomes.

7. The method of claim 1 wherein the composition comprises nanocrystals.

8. The method of claim 1 wherein a catheter is employed to administer the composition.

9. The method of claim 1 further comprising applying ultrasound to the bladder.

10. The method of claim 1 further comprising applying electromagnetic radiation to the bladder.

11. The method of claim 1 further comprising applying a surfactant to the bladder.

12. The method of claim 1 wherein the mammal is a human.

13. The method of claim 1 wherein the mammal has elevated numbers of mast cells.

14. The method of claim 1 wherein the mammal has elevated levels of neurokinin in the urine.

15. The method of claim 1 wherein the mammal is post-transurethral resection.

16. A method to inhibit or treat superficial bladder cancer in a mammal, comprising administering intravescicularly to a mammal having superficial bladder cancer an effective amount of a composition comprising a TLR7 agonist in conjunction with a treatment to enhance local concentrations of the agonist in the bladder mucosa.

17. The method of claim 16 wherein the treatment comprises applying ultrasound to the bladder.

18. The method of claim 16 wherein the treatment comprises applying electromagnetic radiation to the bladder.

19. The method of claim 16 wherein the treatment comprises applying a surfactant to the bladder.

20. The method of claim 16 wherein the mammal is a human.

21. The method of claim 16 wherein the mammal has elevated numbers of mast cells.

22. The method of claim 16 wherein the mammal has elevated levels of neurokinin in the urine.

23. The method of claim 16 wherein the mammal is post-transurethral resection.

24. The method of claim 1 wherein the TLR agonist is formulated as a salt of an acid selected from the group consisting of hydrochloric acid, hydrobromic acid, sulfuric acid, sulfamic acid, phosphoric acid, nitric acid, acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid and isethionic acid.

25. The method of claim 16 wherein the TLR agonist is formulated as a salt of an acid selected from the group consisting of hydrochloric acid, hydrobromic acid, sulfuric acid, sulfamic acid, phosphoric acid, nitric acid, acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid and isethionic acid.
